(19) United States
(12) Patent Application Publication
(10) Pub. No.: US 2026/0087561 A1
(43) Pub. Date: Mar. 26, 2026

(54) SYSTEM AND METHOD FOR AUTOMATED GENERATION OF APPEALS LETTERS

(71) Applicant: Mainsail Parent, LLC, Columbus, GA (US)

(72) Inventors: Chenwei Wang, Chicago, IL (US); Jun Liu, Cary, NC (US); John Sigman, Cary, NC (US); Ya Xue, Durham, NC (US); Spencer Allee, Chicago, IL (US); James C. Bohnsack, Dallas, TX (US); Nick Giannasi, Truckee, CA (US)

(21) Appl. No.: 19/335,414

(22) Filed: Sep. 22, 2025

Related U.S. Application Data
(60) Provisional application No. 63/698,863, filed on Sep. 25, 2024.

(51) Int. Cl. G06Q 40/08; G06F 40/166; G06F 40/205; G06F 40/279; G16H 10/60; G16H 70/20

(52) U.S. Cl. CPC G06Q 40/084; G06F 40/166; G06F 40/205; G06F 40/279; G16H 10/60; G16H 70/20

(57) ABSTRACT

In one or more arrangements, a system is presented for automated processing and/or review of documents for denied benefit system claims and generation of letters to petition for reconsideration. In one or more arrangements, the system includes a front end system and a back end system that is communicatively connected to the front end system. The back end system is configured to perform one or more processes to automatically review documents for a denied benefit system claim and automatically generate a letter to petition for reconsideration. The front end system is configured to provide a user interface for a user to submit a denial letter for the denied benefit system claim and prompt back end system to perform the one or more processes to generate the letter to petition for reconsideration.

### CROSS REFERENCE TO RELATED PATENT APPLICATIONS

This application claims priority to U.S. Provisional Patent Application No. 63/698,863, titled “SYSTEM AND METHOD FOR AUTOMATED GENERATION OF APPEALS LETTERS” filed on Sep. 25, 2024, the entirety of which is hereby incorporated by reference herein.

### FIELD OF THE DISCLOSURE

The disclosure generally relates to systems for processing submissions and responses in benefit systems.

### OVERVIEW OF THE DISCLOSURE

Preparation and processing of submissions in benefit systems is a particularly expensive and time consuming process, often requiring manual review of denied submissions to identify deficiencies/errors prompting the denial, retrieval of supplemental documentation that may be needed, and preparation of appeals letters to petition for reconsideration.

Therefore, for all the reasons stated above, and the reasons stated below, there is a need in the art to improve processes and systems for preparation and processing of benefit system claims. It is an object of the disclosure to provide a system to facilitate automated processing and review of documents for denied benefit system claims to facilitate automated preparation of letters to petition for reconsideration.

These and other objects, features, or advantages of the disclosure will become apparent from the specification, figures, and claims.

### BRIEF DESCRIPTION OF THE DRAWINGS

FIG. 1 shows a diagram of a system for processing and/or review of documents for denied benefit system claims and/or generation of letters, in accordance with one or more arrangements.

FIG. 2 shows a flowchart of an example process performed for generating an appeal letter for a denied benefit claim, in accordance with one or more arrangements.

FIG. 3 shows a flowchart of an example process for retrieval of content by content retrieval subsystem, in accordance with one or more arrangements.

FIG. 4 shows a flowchart of an example process performed by a clinical evaluation subsystem, in accordance with one or more arrangements.

FIG. 5 shows a flowchart of an example process performed by a procedural evaluation subsystem, in accordance with one or more arrangements.

FIG. 6 shows a flowchart of an example process for automated generation of an appeal letter by an appeal letter generation subsystem, in accordance with one or more arrangements.

FIG. 7 shows a flowchart of an example process for automated review of an appeal letter by quality and compliance subsystem, in accordance with one or more arrangements.

FIG. 8 shows a flowchart of a process performed by an LLM subsystem, in accordance with one or more arrangements.

FIG. 9 shows a diagram of an example data processing system that may be used to implement systems, circuits, components, and/or processes of back end system and front end systems, in accordance with one or more arrangements.

FIG. 10 shows a screenshot of an example user interface for human review of draft appeal letters and related information, in accordance with one or more arrangements; the view showing a Draft tab of the user interface.

FIG. 11 shows a screenshot of a Summary tab of the user interface shown in FIG. 10, in accordance with one or more arrangements.

FIG. 12 shows a screenshot of a Denial tab of the user interface shown in FIG. 10, in accordance with one or more arrangements.

FIG. 13 shows a screenshot of a Denial tab of the user interface shown in FIG. 10, in accordance with one or more arrangements.

FIG. 14 shows a screenshot of a Guidelines tab of the user interface shown in FIG. 10, in accordance with one or more arrangements.

FIG. 15 shows a screenshot of an Evidence tab of the user interface shown in FIG. 10, in accordance with one or more arrangements.

FIG. 16 shows a screenshot of an About tab of the user interface shown in FIG. 10, in accordance with one or more arrangements.

### SUMMARY OF THE DISCLOSURE

In one or more arrangements, a system is presented for automated processing and/or review of documents for denied benefit system claims and generation of letters to petition for reconsideration. In one or more arrangements, the system includes a front end system and a back end system that is communicatively connected to the front end system. The back end system is configured to perform one or more processes to automatically review documents for a denied benefit system claim and automatically generate a letter to petition for reconsideration. The front end system is configured to provide a first user interface for a user to submit a denial letter for the denied benefit system claim and prompt back end system to perform the one or more processes to generate the letter to petition for reconsideration.

In one or more arrangements, the processes performed by the back end system include: evaluating a denial of a benefit system claim and determining reasons for the denial; retrieving guidelines and policies relating to applicability of the determined reasons for the denial; retrieving and processing medical records to gather evidence in support of the petition for reconsideration; generating arguments for the letter including for clinical determination, diagnostic documentation, and/or justification for failure to follow procedural requirements; and generating the letter based on the generated arguments.

### DETAILED DESCRIPTION OF THE DISCLOSURE

In the following detailed description of the embodiments, reference is made to the accompanying drawings which form a part hereof, and in which is shown by way of illustration specific embodiments in which the disclosure may be practiced. The embodiments of the present disclosure described below are not intended to be exhaustive or to limit the disclosure to the precise forms in the following detailed description. Rather, the embodiments are chosen and described so that others skilled in the art may appreciate and understand the principles and practices of the present disclosure. It will be understood by those skilled in the art that various changes in form and details may be made without departing from the principles and scope of the invention. It is intended to cover various modifications and similar arrangements and procedures, and the scope of the appended claims therefore should be accorded the broadest interpretation so as to encompass all such modifications and similar arrangements and procedures. For instance, although aspects and features may be illustrated in or described with reference to certain figures or embodiments, it will be appreciated that features from one figure or embodiment may be combined with features of another figure or embodiment even though the combination is not explicitly shown or explicitly described as a combination. In the depicted embodiments, like reference numbers refer to like elements throughout the various drawings.

It should be understood that any advantages and/or improvements discussed herein may not be provided by various disclosed embodiments, or implementations thereof. The contemplated embodiments are not so limited and should not be interpreted as being restricted to embodiments which provide such advantages or improvements. Similarly, it should be understood that various embodiments may not address all or any objects of the disclosure or objects of the invention that may be described herein. The contemplated embodiments are not so limited and should not be interpreted as being restricted to embodiments which address such objects of the disclosure or invention. Furthermore, although some disclosed embodiments may be described relative to specific materials, embodiments are not limited to the specific materials or apparatuses but only to their specific characteristics and capabilities and other materials and apparatuses can be substituted as is well understood by those skilled in the art in view of the present disclosure.

It is to be understood that the terms such as “left, right, top, bottom, front, back, side, height, length, width, upper, lower, interior, exterior, inner, outer, and the like as may be used herein, merely describe points of reference and do not limit the present invention to any particular orientation or configuration.

As used herein, “and/or” includes all combinations of one or more of the associated listed items, such that “A and/or B” includes “A but not B,” “B but not A,” and “A as well as B,” unless it is clearly indicated that only a single item, subgroup of items, or all items are present. The use of “etc.” is defined as “et cetera” and indicates the inclusion of all other elements belonging to the same group of the preceding items, in any “and/or” combination(s).

As used herein, the singular forms “a,” “an,” and “the” are intended to include both the singular and plural forms, unless the language explicitly indicates otherwise. Indefinite articles like “a” and “an” introduce or refer to any modified term, both previously introduced and not, while definite articles like “the” refer to a same previously introduced term; as such, it is understood that “a” or “an” modify items that are permitted to be previously-introduced or new, while definite articles modify an item that is the same as immediately previously presented. It will be further understood that the terms “comprises,” “comprising,” “includes,” and/or “including,” when used herein, specify the presence of stated features, characteristics, steps, operations, elements, and/or components, but do not themselves preclude the presence or addition of one or more other features, characteristics, steps, operations, elements, components, and/or groups thereof, unless expressly indicated otherwise. For example, if an embodiment of a system is described as comprising an article, it is understood the system is not limited to a single instance of the article unless expressly indicated otherwise, even if elsewhere another embodiment of the system is described as comprising a plurality of articles.

It will be understood that when an element is referred to as being “connected,” “coupled,” “mated,” “attached,” “fixed,” etc. to another element, it can be directly connected to the other element, and/or intervening elements may be present. In contrast, when an element is referred to as being “directly connected,” “directly coupled,” “directly engaged” etc. to another element, there are no intervening elements present. Other words used to describe the relationship between elements should be interpreted in a like fashion (e.g., “between” versus “directly between,” “adjacent” versus “directly adjacent,” “engaged” versus “directly engaged,” etc.). Similarly, a term such as “operatively”, such as when used as “operatively connected” or “operatively engaged” is to be interpreted as connected or engaged, respectively, in any manner that facilitates operation, which may include being directly connected, indirectly connected, electronically connected, wirelessly connected or connected by any other manner, method or means that facilitates desired operation. Similarly, a term such as “communicatively connected” includes all variations of information exchange and routing between two electronic devices, including intermediary devices, networks, etc., connected wirelessly or not. Similarly, “connected” or other similar language particularly for electronic components is intended to mean connected by any means, either directly or indirectly, wired and/or wirelessly, such that electricity and/or information may be transmitted between the components.

It will be understood that, although the ordinal terms “first,” “second,” etc. may be used herein to describe various elements, these elements should not be limited to any order by these terms unless specifically stated as such. These terms are used only to distinguish one element from another; where there are “second” or higher ordinals, there merely must be a number of elements, without necessarily any difference or other relationship. For example, a first element could be termed a second element, and similarly, a second element could be termed a first element, without departing from the scope of example embodiments or methods.

Similarly, the structures and operations discussed herein may occur out of the order described and/or noted in the figures. For example, two operations and/or figures shown in succession may in fact be executed concurrently or may sometimes be executed in the reverse order, depending upon the functionality/acts involved. Similarly, individual operations within example methods described below may be executed repetitively, individually or sequentially, to provide looping or other series of operations aside from single operations described below. It should be presumed that any embodiment or method having features and functionality described below, in any workable combination, falls within the scope of example embodiments.

As used herein, various disclosed embodiments may be primarily described in the context of medical benefit systems. However, the embodiments are not so limited. It is appreciated that the embodiments may be adapted for use in other applications which may be improved by the disclosed structures, arrangements and/or methods. The system is merely shown and described as being used in the context of medical benefit systems for ease of description and as one of countless examples.

### System 10:

With reference to the figures, a system for processing and/or review of documents for denied benefit system claims and/or generation of letters to petition for reconsideration 10 (or simply system 10) is presented. The system 10 is formed of any suitable design, arrangement, and circuitry and is configured to facilitate review and analysis of documents for denied benefit system claims and generation of appeal letters to petition for reconsideration.

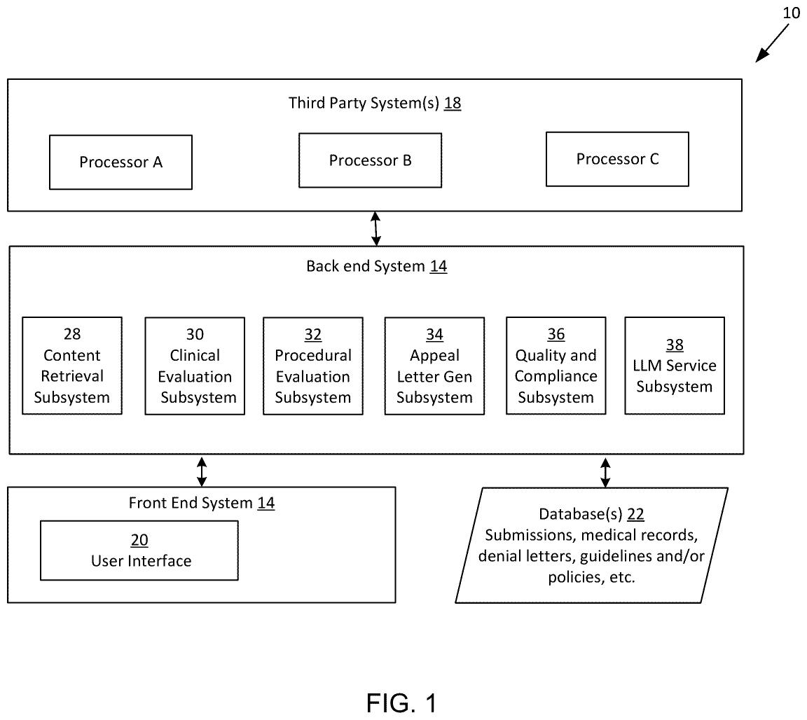

In one or more arrangements, as shown in FIG. 1 for example, the system 10 includes a front end system 14, a back end system 16, and a database 22, among other components. In one or more arrangements back end system 16 is communicatively connected to front end system 14 and to database 22 over one or more data networks.

### Front End System 14:

In one or more arrangements, system 10 includes a front end system 14. Front end system 14 is formed of any suitable size, shape, design, and/or technology and is configured to provide one or more user interfaces for end users to communicate and interact with back end system 16 to facilitate input, access to, and processing of data stored therein and/or perform various operations related to, for example, processing and review of responses (e.g. denial letters) from third party systems 12, identification of defects in claims and/or other reasons for denial, and/or generation of appeal letters to address identified defects and/or reasons for denial and petition for reconsideration. In one or more arrangements, system 10 includes multiple user interfaces for use by the different types of end-users. In some various different arrangements shown, front end systems 16 includes respective interfaces for processors, doctors, patients, and/or any other relevant party.

### User Interface 20:

User interface 20 provided by front end system 14 may be formed of any suitable size, shape, and design, and/or technology and is configured to permit end users to interact with back end system 16 to facilitate input, access to, and processing of relevant data to uploading of or access to documents for denied benefit system claims and/or utilization of various processes and/or systems of back end system 16 to facilitate processing and/or review of documents for denied benefit system claims and/or generation of letters to petition for reconsideration.

While the arrangements may be primarily described with reference to system 10 having front end system 14 for users to interact with back end system 16, the arrangements are not so limited. Rather, it is contemplated that in some various arrangements, back end system 16 may be configured to perform one or more processes described herein for generation of appeals letters automatically without user input and front end system 14 may be omitted.

### Database 22:

Database 22 is formed of any suitable size, shape, design and is configured to facilitate storage and retrieval of data. In the arrangement shown, as one example, database 22 is local data storage connected to backend system 16 (e.g., via a data bus or electronic network). However, embodiments are not so limited. Rather, it is contemplated that in one or more arrangements, database 22 may be remote storage or cloud based service communicatively connected to backend system 16 via one or more external communication networks. In one or more arrangements,

### Back End System 16:

Back end system 16 is formed of any suitable size, shape, design, and/or technology and is configured to perform one or more functions to facilitate automated review and/or analysis of documents for denied benefit system claims and automated generation of appeal letters to petition for reconsideration.

In some various arrangements, such analysis and generation may include but are not limited to, for example, automated processing and review of responses (e.g. denial letters) from third party systems 12, identification of defect in claims and/or other reasons for denial, and/or generation of supplemental claims (e.g., appeal letters) to address identified defects and/or reasons for denial and petition for reconsideration.

In the arrangement shown, as one example, back end system 16 includes a clinical evaluation subsystem 30, a procedural evaluation subsystem 32, a criteria subsystem 32, a content retrieval subsystem 32, an appeal letter generation subsystem 34, a quality and compliance subsystem 36, and an LLM service subsystem 38, among other components.

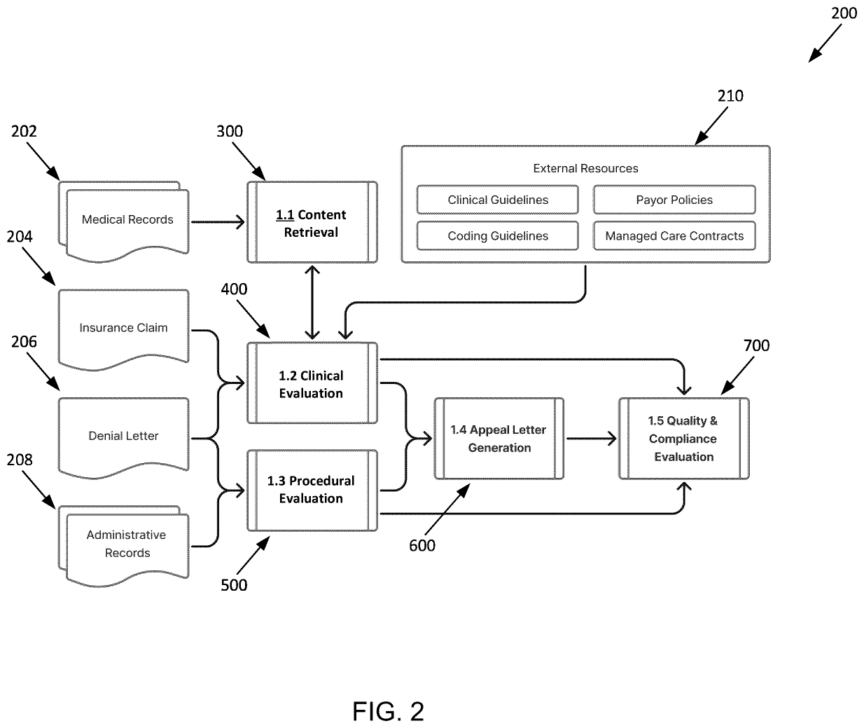

FIG. 2 shows a flowchart of an example process 200 performed by backend system 16 using the various subsystems to generate an appeal letter for a denied benefit claim, in accordance with one or more arrangements. In this illustrative example, the process receives/retrieves as inputs: medical records 202, insurance claim data 204, the denial letter 206, and administrative records 208. At process block 300, content retrieval subsystem 32 performs a content retrieval process to dissect the medical records 202 and make the data therein searchable for use by analytics processes (e.g., languages learning machines). At process block 400, clinical evaluation subsystem 32 evaluates the insurance claim data 204, denial letter 206, the searchable medical record data provided by content retrieval subsystem 32, and/or various external resources 210 (e.g., clinical guidelines, payor policies, coding guidelines, managed care contracts, etc.) to determine if the services performed were medically necessary. At process block 500, procedural evaluation system 30 evaluates the denial letter 206 and administrative records 208 to determine if proper procedures were followed for the performed services (e.g., notice, authorizations, etc.). At process block 600, appeal letter generation subsystem 34 generates an appeal letter using data determined in the clinical evaluation 400 and procedural evaluation 500 processes. Following appeal letter generation, quality and compliance subsystem 36 evaluates the generated appeal letter at process block 700 and determines if it is suitable for claim to the carrier for appeal.

### Content Retrieval Subsystem 28:

Content retrieval subsystem 28 is formed of any suitable size, shape, design, and/or technology and is configured to perform various operations to retrieve and process documents to facilitate searching for information for analytics processes of clinical evaluation subsystem 30 and/or procedural evaluation subsystem 32.

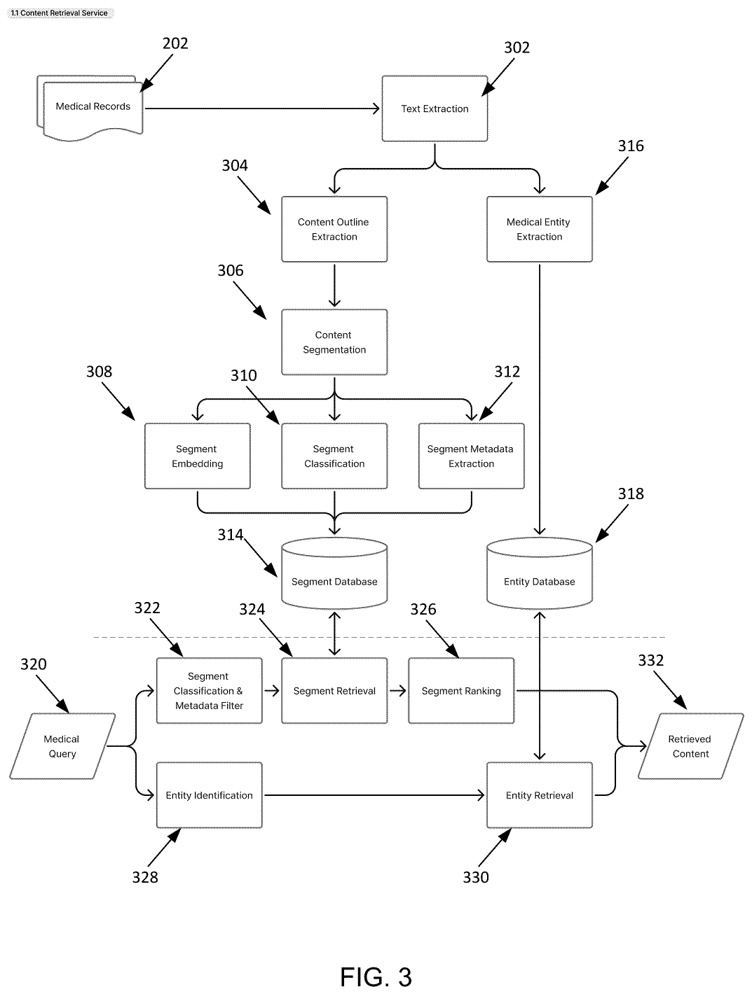

FIG. 3 shows a flowchart of an example process for retrieval of content by content retrieval subsystem 28, in accordance with one or more arrangements. In this example arrangement, the medical record 202 and/or other related documents are processed for storage in one or more databases (e.g., 314/318). At process block 302 text is extracted from medical records 202 and/or other documents retrieved from databases 22 and/or one or more other data sources 210 for example, using optical character recognitional. In this example, the extracted text describes various information placed in the record for the patient in the course of one or more provider visits/stays relating to the denied claim, is organized into a searchable form for storage for use by analytics processes. Such information may include but is not limited to notes of doctors, staff, labs, tests results/data, treatments given, etc. In this example, sections outlining the content are determined at process block 306 at process block 304 and content is divided into segments at process block 306.

Various arrangements may apply various different techniques to process, organize and format the segments for use by analytics processes. In this example arrangement, the segments are embedded into a data format at process block 308 and classified at process block 310. Meta data is extracted from the content segments at process block 312. The content segments and metadata are stored in segment database 314.

Additionally, at process block 316, medical entity information is extracted from the text extracted at process block 302 and stored in entity database 318. Such entity information may include but is not limited to, for example, who the patient is, what labs/test did they have, what diagnoses were recorded, etc.

Once data is stored in databases 314/318, the data may be queried as needed by analytics or other processes.

In this example, when a medical query 320 is received, process block 322 determines from the query which metadata fields in the content database 314 are applicable. At process block 324, the determined metadata fields are used to retrieve segments relating to the query 320 are retrieved from database 314. In one or more arrangements, process block 324 is configured to retrieve segments that are similar or related to the query. As an illustrative example, for a query asking if a patient was diagnosed with lung cancer prior to a specified date, process block 324 may retrieve any segments that is generally related to the respiratory system and prior to the specified date. At process block 326, retrieved segments are ranked to determine which segment(s) are most relevant to the query.

At process block 328, the process determines if the query pertains to any structured entities that would be stored in database 318. If so, data for the entities is retrieved at process block 330 and combined with the higher ranked segment(s) from process block 326 as the retrieved content 332.

### Clinical Evaluation Subsystem 30:

In one or more arrangements, back end system 16 includes a clinical evaluation subsystem 30. Clinical evaluation subsystem 30 is formed of any suitable size, shape, design, and/or technology and is configured to perform various analytics processes to evaluate clinical aspects of a denied benefit system claim and/or derive additional data metrics and/or content for use by various other subsystems of back-end system 16.

In one or more arrangements, the analytics processes performed by clinical evaluation subsystem 30 are configured to process information received from front end system 14 and/or data stored in database 22 to derive additional data metrics pertinent to denied benefit system claims.

In some various embodiments, the analytics processes may include various guided and/or unguided artificial intelligence and/or machine learning techniques including, but not limited to: neural networks, genetic algorithms, support vector machines, k-means, kernel regression, discriminant analysis and/or various combinations thereof. In different implementations, analysis may be performed locally, remotely, or a combination thereof.

In an example arrangement, the analytics processes utilize one or more machine learning algorithms that are trained to implement a large language model (LLM) and/or a generative artificial intelligence model, which is used by various subsystems of back-end system 16 to evaluate, summarize and/or generate content related to clinical assessment of a denied benefit system claim. In some arrangements, various subsystems of back-end system 16 are configured to evaluate data for respective cases and dynamically create appropriate prompts based on such evaluation to facilitate respective operation of the subsystems.

However, the arrangements are not so limited. Rather, it is contemplated that in some various arrangements, subsystems of back-end system 16 may utilize various additional or alternative types of artificial intelligence. Moreover, although clinical evaluation subsystem 30 may be depicted as being implemented locally alongside other subsystems of backend system 16, it is contemplated that in some various arrangements, clinical evaluation subsystem 30 may be implemented remotely and/or be provided by one or more third party systems.

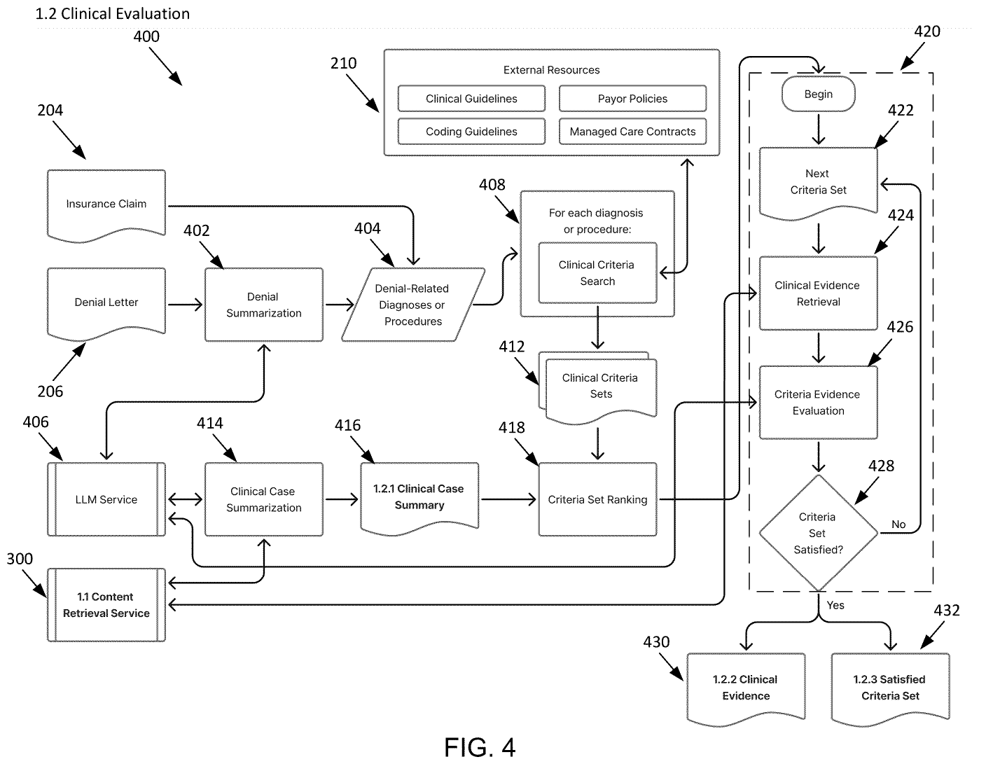

FIG. 4. shows a flowchart of an example process 400 for clinical evaluation that may be performed by clinical evaluation subsystem 30 in accordance with one or more arrangements.

In this example, process 400 evaluates a denial letter 206 and insurance claim 204 to determine sets of criteria 412 that may show that a procedure or treatment was medically necessary and then evaluates the determined sets of criteria 412 to select the set of criterial 432 that best supports a conclusion that the procedure or treatment was medically necessary.

In this illustrative example, the process 400 receives/retrieves as inputs: insurance claim data 204, the denial letter 206, and/or other data in external resources 210. At process block 402, the denial letter 206 is summarized (e.g., using LLM service 406). At process block 404, the summary is evaluated with structured data from the insurance claim 204 to identify diagnoses and/or procedures performed that are related to the denial. For instance, in one or more arrangements, at process block 404 codes that are relevant to the denial are extracted from the denial letter, insurance claims 204, and/or are input by a user. At process block 408, for each identified diagnosis or procedure, the process searches relevant guidelines, policies, contracts (e.g., using the identified codes) to identify any set(s) 412 of clinical criteria that indicate that the diagnosis and/or procedure was correct and medically necessary.

In conjuncture with determining clinical criterial sets 412, at process 414, the process 400 content retrieval service 300 is used to retrieve content from medical records and/or other documents and summarize the retrieved content (e.g., using LLM service 406) to produce a clinical case summary 416. At process block 418, the clinical case summary 416 is used to rank the determined clinical criteria sets 412 (e.g., according to which sets best support the diagnoses and/or procedures related to the reasons for denial).

Following sorting of the determined criteria sets 412, a sub-process 420 is performed to identify the strongest of clinical criteria sets 412 that is satisfied by the clinical case records. In this illustrative example, process 420 operates in an iterative loop. In each iteration, the next best criteria set 422 is selected from the ranked clinical criteria sets 412. At process block 424 clinical evidence needed to evaluate the selected criteria set 422 is retrieved (e.g., using content retrieval service 300). At process block 426, the retrieved clinical evidence is evaluated along with the selected criteria set 422. If the selected criteria set 422 is not satisfied at decision block 428, the process 420 is repeated for the next criteria set in the ranked criteria sets. The process 420 repeats in this manner until a selected criteria set 422 is satisfied at decision block 428. The clinical evidence 430 and satisfied criteria set 432 are returned for use in generation of an appeal letter.

By identifying, ranking, and evaluating criteria sets 412 in this manner, the process 400 can quickly and efficiently consider alternative justifications that may not have been used in the initial insurance claim 204, and determine the set of criteria that is satisfied and that best supports that the diagnosis and treatment were correct and medically necessary. For instance, once a criteria set that is satisfied by the clinical evidence has been found, since retrieval and evaluation of evidence is not performed for the remaining sets of criteria, the time, energy, and resources for such retrieval and evaluation is avoided.

### Procedural Evaluation Subsystem 32:

Procedural evaluation subsystem 32 is formed of any suitable size, shape, design, and/or technology and is configured to perform various and/or analytics processes to evaluate procedural aspects of a denied benefit system claim and/or derive additional data metrics.

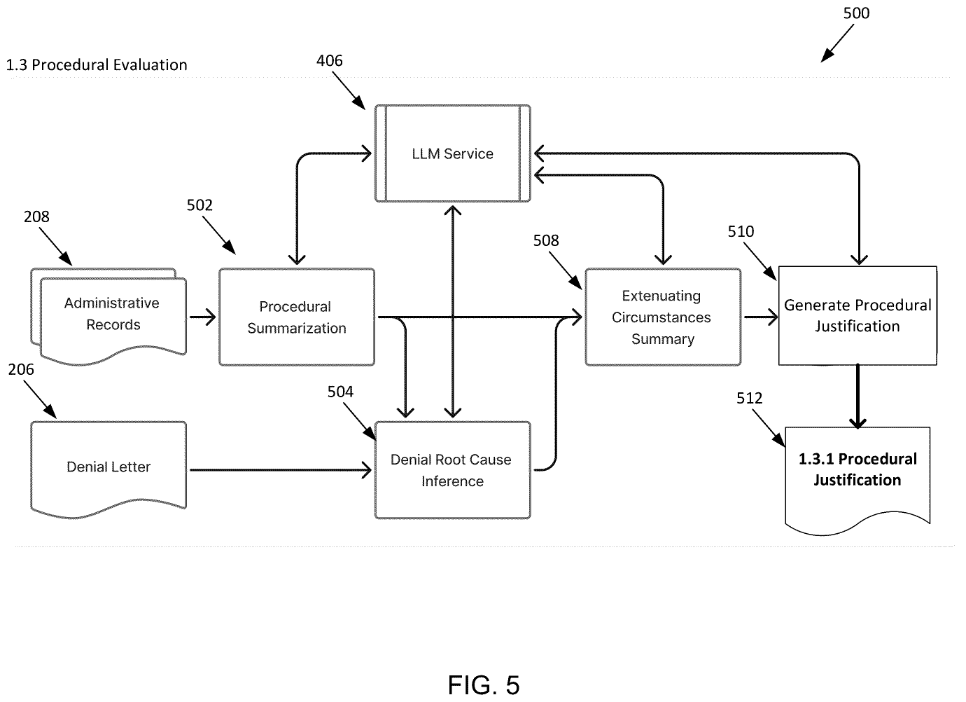

FIG. 5 shows a flowchart of an example process 500 performed by procedural evaluation subsystem 32, in accordance with one or more arrangements. In this illustrative example, the process 500 receives administrative records 208 and the denial letter 206 initial inputs. The denial letter 206 and administrative records are ingested into the system 10, for example, by a user uploading the letter via front end system 14 or by automatically retrieving the letter from a database(s) or other data source.

In this example, at process block 502, procedures relating to the claim are summarized from administrative records 208 (e.g., using LLM service 406). As an illustrative example, such procedures may describe the administrative account of the administrative steps taken and circumstances relating to the intake/diagnosis/treatment of the patient (e.g., intake/admission, authorizations submitted to insurance providers, responses from insurance provides, procedural reasons for not getting authorizations, etc.). At process block 504, the denial letter 206 is evaluated along with the procedural summary to infer the root cause for the denial (e.g., using LLM service 406).

If inferred root cause for denial is failure to follow one or more procedural requirements, extenuating circumstances indicating reasons why such procedural requirements could not have been followed are summarized at process block 508 (e.g., using LLM service 406). At process block 510, a procedural justification is generated (e.g., using LLM service 406),

### Appeal Letter Generation Subsystem 34:

Appeal letter generation subsystem 34 is formed of any suitable size, shape, design, and/or technology and is configured to perform various operations and/or analytics processes to facilitate automated generation of an appeal letter for a denied benefit system claim.

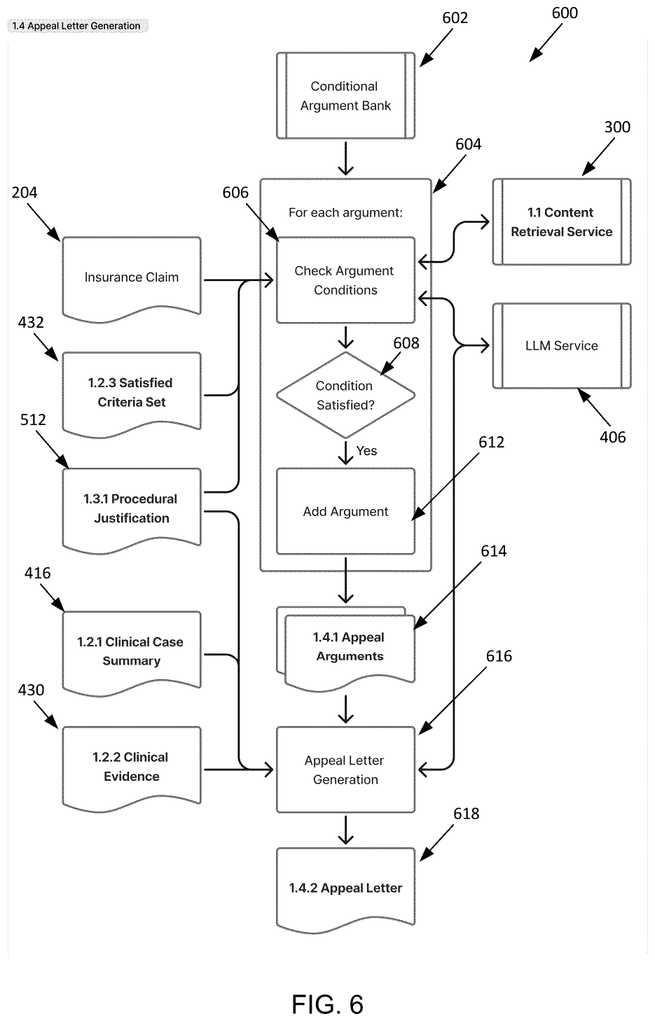

FIG. 6 shows a flowchart of an example process 600 for automated generation of an appeal letter by appeal letter generation subsystem 34, in accordance with one or more arrangements. In this example, a plurality of arguments specified in a conditional arguments bank 602 are evaluated at process block 604 for potential use in the appeal letter to be generated. In one or more arrangements, each argument in conditional arguments bank 602 specifies a respective set of conditions that should be satisfied if the argument is to be included in the appeal letter.

In this example, for each argument process block 606 evaluates criteria specified for the argument using information in insurance claim 204, the satisfied criteria set 432, the procedural justification 512, and/or additional information retrieved relating to the criteria (e.g., using content retrieval service 300). In one or more arrangements, process block 606 may use LLM service 406 in evaluating if one or more conditions are satisfied. If the criteria are satisfied at decision block 608, the argument is added to a list of arguments 614 for appeal at process block 612. If the criteria are not satisfied at decision block 608, the argument is not included.

After arguments have been evaluated at process block 604, an appeal letter 618 is generated at process block 616. In this example, process block 604 generates an appeal letter 618 using information in the procedural justification 512, the clinical case summary 416, and/or the clinical evidence 430 along with the selected list of appeal arguments 614. In one or more arrangements, process block 616 may utilize LLM service 406 and/or templates to facilitate generation of text for the appeal letter.

In one or more arrangements, the arguments in conditional argument bank 602 have respective boilerplate and/or template language to be added to the appeal letter with field(s) for insertion of metadata from the procedural justification 512, the clinical case summary 416, the clinical evidence 430, and/or other data sources. Additionally or alternatively, in some arrangements the arguments in conditional argument bank 602 may have respective prompts for generation of text for one or more arguments using LLM service 406.

### Quality and Compliance Subsystem 36

Quality and compliance subsystem 36 is formed of any suitable size, shape, and design, and is configured to automatically review the generated appeal letter 618 and determine if human review and/or revision is required.

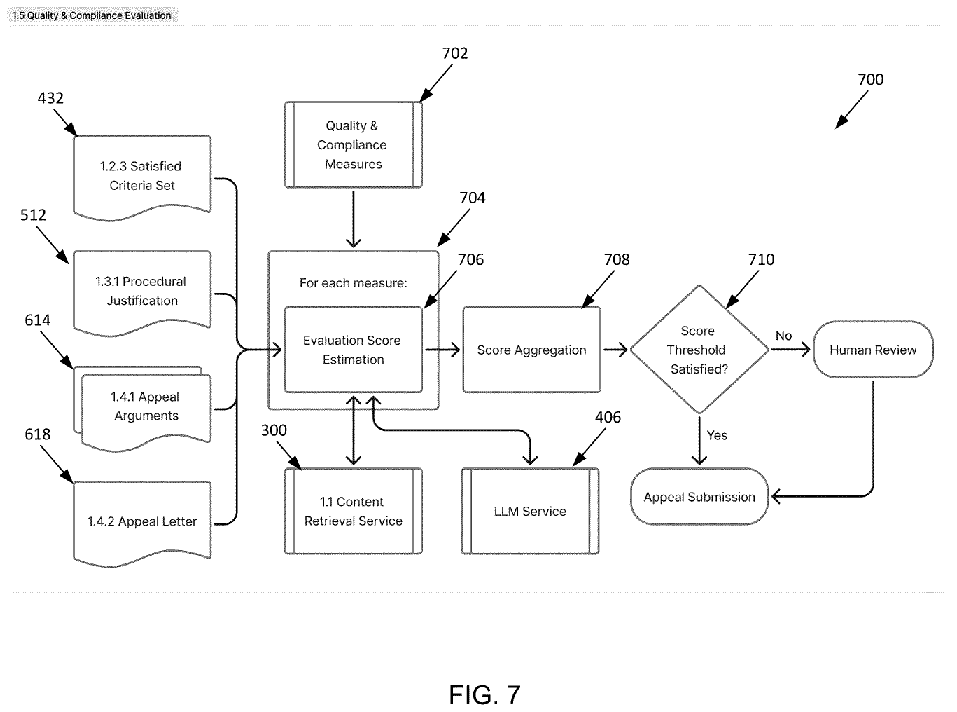

FIG. 7 shows a flowchart of an example process 700 for automated review of an appeal letter by quality and compliance subsystem 36, in accordance with one or more arrangements.

In this example, the appeal letter 618 is evaluated at process block 704 according to a predefined set of quality and compliance measures 702.

For each such measure, the appeal letter 618 is evaluated and scored for the quality and compliance measures at process block 706. In one or more arrangements, in performing the evaluation, process block 606 may utilize information in satisfied criteria set 432, procedural justification 512, appeal arguments 614, and/or other data sources (e.g., via content retrial service 300) to assess the quality and compliance measures. In one or more arrangements, process block 706 may utilize LLM service 406 (or other AI process) to evaluate and/or score quality and compliance measures.

At process block 708, the scores for the various quality and compliance measures are aggregated. If the aggregate score is above a predetermined threshold at decision block 710, the process 700 causes the appeal letter 618 to be submitted to the insurance provider according to the designated method for appealing the denial letter (e.g., by electronic submission, email, and/or mailing a paper letter). If the aggregate score is below a predetermined threshold at decision block 710, process 700 causes the appeal letter 618 to be flagged and/or forwarded for human review and/or revision.

### LLM Service Subsystem 38:

LLM service subsystem 38 is formed of any suitable size, shape, and design and is configured to use artificial intelligence to facilitate evaluation of data and/or generation content in support of various processes performed by system 10.

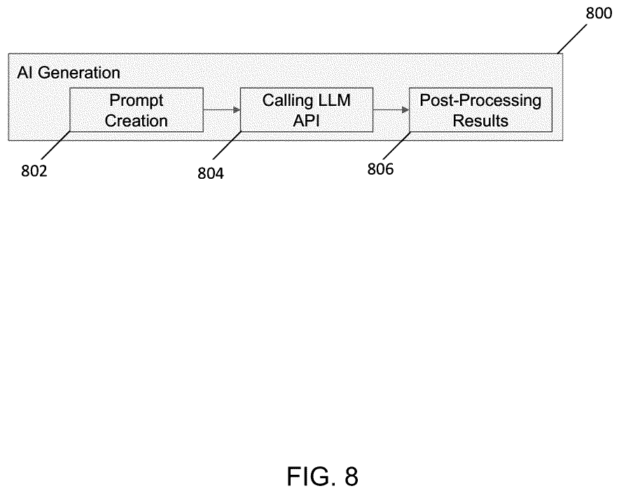

In one or more arrangements, LLM service subsystem 38 may be implemented using the AI generation process 800 shown in FIG. 8. In this example, process 800 begins at process block 802 with creation of a prompt to be provided to the LLM of clinical evaluation subsystem 30. In one or more arrangements, the prompt is generated using a template specific to the subsystem or process using LLM Service Subsystem 38 (e.g., content retrieval subsystem 28, clinical evaluation subsystem 30, procedural evaluation subsystem 32, or appeal letter generation subsystem 34)

Additionally or alternatively, in one or more arrangements, the prompt may be dynamically created and/or adjusted based on a ruleset utilizing one or more parameters in the extracted content or other records related to the denied benefit system claim. Additionally or alternatively, in one or more arrangements, the AI generation process 802 may utilize various prompt engineering techniques to create/adjust prompts including but not limited to, for example, chain of thought prompting, tree of thought prompting, Maieutic prompting, complexity based prompting, least to most prompting, self-refine prompting, directional stimulus prompting, zero shot prompting, active prompt, and/or any other prompt engineering techniques.

After prompt creation at process block 802, the process proceeds to process block 804, where the prompt is input to the LLM (or another AI algorithm), for example using an API for the LLM. At process block 806, post processing is performed on results from the LLM (e.g., to adjust, package, and/or export results to the calling process or subsystem).

While various arrangements may be described as using prompts to make calls to one LLM, the arrangements are not so limited. Rather, it is envisioned that in some arrangements, system 10 may utilize several different LLMs (or other AI) that are trained for performance of specific tasks related to the evaluation of denied claims and generation of appeals letters. Furthermore, the arrangements are not limited to use of LLMs. Rather, it is envisioned that in some various arrangements subsystem 38 and/or various processes or subsystems of system 10 may additionally or alternatively utilize various guided and/or unguided artificial intelligence and/or machine learning techniques including, but not limited to: neural networks, genetic algorithms, support vector machines, k-means, kernel regression, discriminant analysis and/or various combinations thereof.

At process block 712, an AI generation process is performed to prompt a LLM or other AI algorithm to automatically reflect and/or evaluate the draft clinical determination arguments and generate feedback. At process block 714, an AI generation process is performed to regenerate a second draft of the clinical determination arguments using the loaded evidence, case summary, and guidelines as well as the feedback generated at process block 712. In this example, an AI generation process for generation of arguments for diagnostic documentation is performed at process block 716 using the loaded evidence and case summary. In this example, an AI generation process for generation of arguments for guideline justification is performed at process block 718 using the denial summer. At process block 720, arguments generated for clinical determination, diagnostic documentation, and guideline justification are serialized.

### Third Party System(s) 18:

In one or more arrangements, back end system 16 is configured to communicate benefit system claims and/or appeal letters to and receive responses from various third party systems 18, which are configured to receive and process benefit system claims.

In one or more arrangements, back end system 16 is configured to access and communicate with third party system(s) systems 18 using respective application program interfaces (APIs). However, the arrangements are not so limited. Rather, it is contemplated that in some various arrangements, back end system 16 is configured to communicate with third party suppliers using various means and methods including but not limited to, for example, data third party provided APIs, custom APIs, SQL queries, file downloads, via email, fax, SFTP, Dropbox, and/or any other means or methods for communication of data.

### Data Processing System 900:

Various blocks, modules, or other circuits of the back end system 16 and front end system 14 may be implemented to carry out one or more of the operations and activities described herein and/or shown in the figures. In these contexts, a “block” (also sometimes “logic circuit,” “control circuit,” “processing circuit,” “server,” “module,” “data processing system” or “system”) is a circuit specifically configured and arranged to carry out one or more of these or related operations/activities. For example, such circuits may be discrete logic circuits or programmable logic circuits configured and arranged for implementing these operations/activities, as shown in the figures and/or described in the specification. In certain embodiments, such a programmable circuit may include one or more programmable integrated circuits (e.g., field programmable gate arrays and/or programmable ICs). Additionally or alternatively, such a programmable circuit may include one or more processing circuits (e.g., a computer, tablet, microcontroller, system-on-chip, smart phone, server, and/or cloud computing resources). For instance, computer processing circuits may be programmed to execute a set (or sets) of instructions (and/or configuration data). The instructions (and/or configuration data) can be in the form of firmware or software stored in and accessible from a memory (circuit). Certain aspects are directed to a computer program product (e.g., nonvolatile memory device), which includes a machine or computer-readable medium having stored thereon instructions which may be executed by a computer (or other electronic device) to perform these operations/activities.

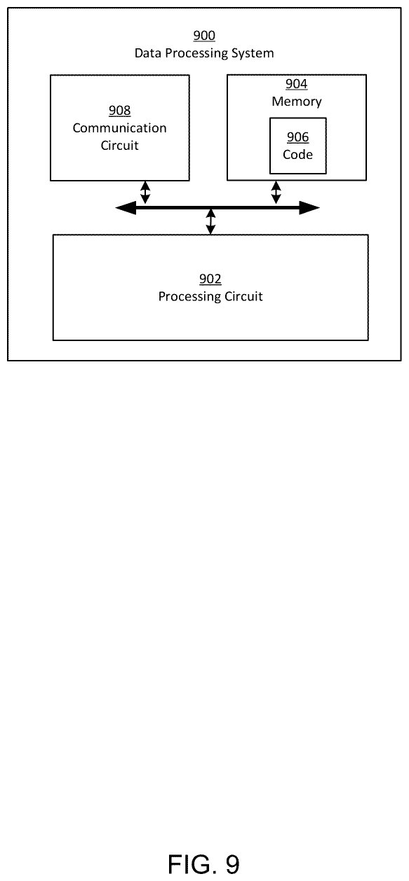

FIG. 9 shows an example data processing system 900 that may be used to implement systems, circuits, components, and/or processes of back end system 16 and front end systems 16, in accordance with one or more arrangements. Data processing system 900 is formed of any suitable size, shape, design, and/or technology and is configured to carry out the one or more of these or related operations/activities described herein. In the arrangement shown, as one example, data processing system 900 includes a processing circuit 902 and memory 904 having software code 906 or instructions that facilitates the processing and/or display of information, and a communication circuit 908, among other components.

Processing circuit 902 may be any computing device that receives and processes information and outputs commands according to software code 906 or instructions stored in memory 904. Memory 904 may be any form of information storage such as flash memory, ram memory, dram memory, a hard drive, or any other form of memory. Processing circuit 902 and memory 904 may be formed of a single combined unit. Alternatively, processing circuit 902 and memory 904 may be formed of separate but electrically connected components. Alternatively, processing circuit 902 and memory 904 may each be formed of multiple separate but electrically connected components.

Software code 906 or instructions is any form of information or rules that direct processing circuit 902 how to receive, interpret, and respond to information to operate as described herein. Software code 906 or instructions is stored in memory 904 and accessible to processing circuit 902. As an illustrative example, in one or more arrangements, software code or instructions may configure processing circuit 902 to interact with users via front end system 14 and perform various processes in response to user input.

Communication circuit 908 is formed of any suitable size, shape, design, and/or technology and is configured to facilitate communication with various other components of system 14 (as may be applicable). In one or more arrangements, as one example, communication circuit 908 includes a transceiver circuit and an antenna. A transceiver is any electronic device that facilitates two-way communication, that is, the delivery of information between data processing system 900 and other components of system 10. An antenna is any device that is configured to receive wireless signals from over-the-air communication and/or transmit wireless signals in over-the-air communication. In an example arrangement, a transceiver of communication circuit 908 is connected with a respective antenna, which may be a monopole antenna, dipole antenna, a loop antenna, a fractal antenna, or any other form of an antenna, to facilitate transmission and/or reception of signals in the form of electromagnetic radio frequencies. Additionally or alternatively, the transceiver of communication circuit 908 may be configured to communicate over a wired communication channel.

In various arrangements, communication circuit 908 may be configured to communicate with various components of system 10 using various wired and/or wireless communication technologies and protocols over various networks and/or mediums including but not limited to, for example, Serial Data Interface 12 (SDI-12), UART, Serial Peripheral Interface, PCI/PCIe, Serial ATA, ARM Advanced Microcontroller Bus Architecture (AMBA), USB, Firewire, RFID, Near Field Communication (NFC), infrared and optical communication, 802.3/Ethernet, 802.11/WIFI, Wi-Max, Bluetooth, Bluetooth low energy, Ultra Wideband (UWB), 802.15.4/ZigBee, ZWave, GSM/EDGE, UMTS/HSPA+/HSDPA, CDMA, LTE, 4G, 5G, FM/VHF/UHF networks, and/or any other communication protocol, technology or network.

Although in some arrangements, various circuits, components, systems, programs, or processes of back end system 16, front end system 14, or other portions of system 10 may be primarily described or shown as being implemented together on the same system, machine, network, program or process, the arrangements are not so limited. Rather it is contemplated that such components, systems, programs, or processes of back end system 16, front end system 14 or other portions of system 10 may be implemented separately on by separate processes or programs and/or on separate circuits, systems, and/or components on the same bus or network or communicatively connected between different networks. Conversely, although in some arrangements, various circuits, components, systems, programs, or processes of back end system 16, front end system 14, or other portions of system 10 may be primarily described or shown as being implemented separately, the arrangements are not so limited. Rather, it is contemplated that such components, systems, programs, or processes of back end system 16, front end system 14, and/or other portions of system 10 may be implemented together by the same processes or program and/or on the same circuit, system, and/or component of system 10.

In one or more arrangements, data processing system 900 may additionally include one or more application specific integrated circuits (ASICs) (not shown) to facilitate local implementation of an LLM or other artificial intelligence process. For example, in one or more arrangements, data processing system 900 may include an ASIC configured to implement an artificial neural network (ANN) that is trained to provide an LLM or other artificial intelligence process. As an illustrative example, in some arrangements the ACIS includes a plurality of neurons organized in an array, wherein each neuron comprises a register, a microprocessor, and at least one input; and a plurality of synaptic circuits, each synaptic circuit including a memory for storing a synaptic weight, wherein each neuron is connected to at least one other neuron via one of the plurality of synaptic circuits. In one or more arrangements, the synaptic circuits are trained to implement an LLM used by LLM service subsystem 38 as described herein.

### User Interface 20:

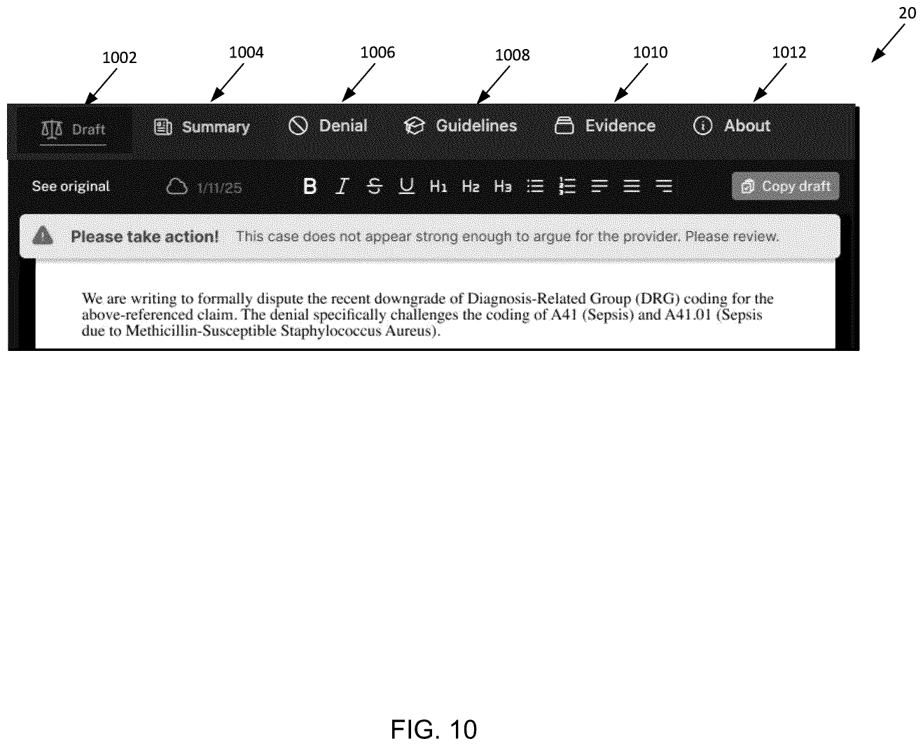

FIGS. 10-16 show screenshots of an example user interface 20 for human review of draft appeal letters and related information, in accordance with one or more arrangements. In this illustrative example, the user interface 20 has six navigation tabs: Draft 1002, Summary 1004, Denial 1006, Guidelines 1008, Evidence 1010, and About 1012.

FIG. 10 shows a screenshot of the Draft tab 1002 of the example user interface 20. In this tab 1002, a user can read through the draft appeal letter generated for appeal of the denied benefit claim.

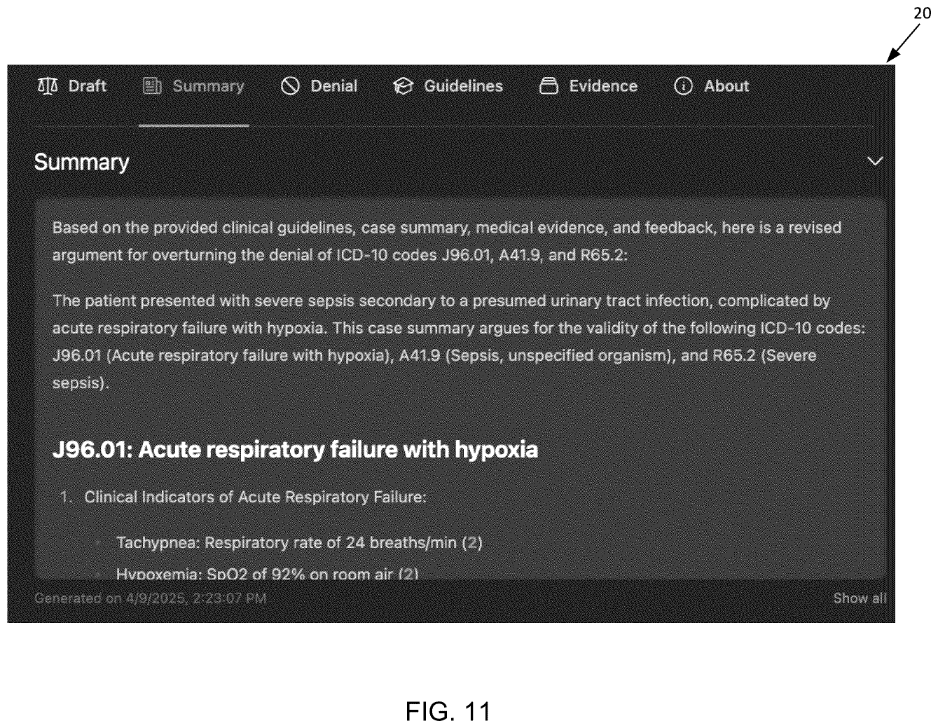

FIG. 11 shows a screenshot of the Summery tab 1004 of the example user interface 20. The Summary tab 1004, provides an overview of the appeal with citations to the medical record so a review can validate the details of the arguments. The user can also find other key information like admission dates, arguments used and admitting diagnosis depending on your denial type. To check a citation, the user selects a highlighted page number next to the value they would like to check. This will open the medical record and navigate to the page where we gathered that information.

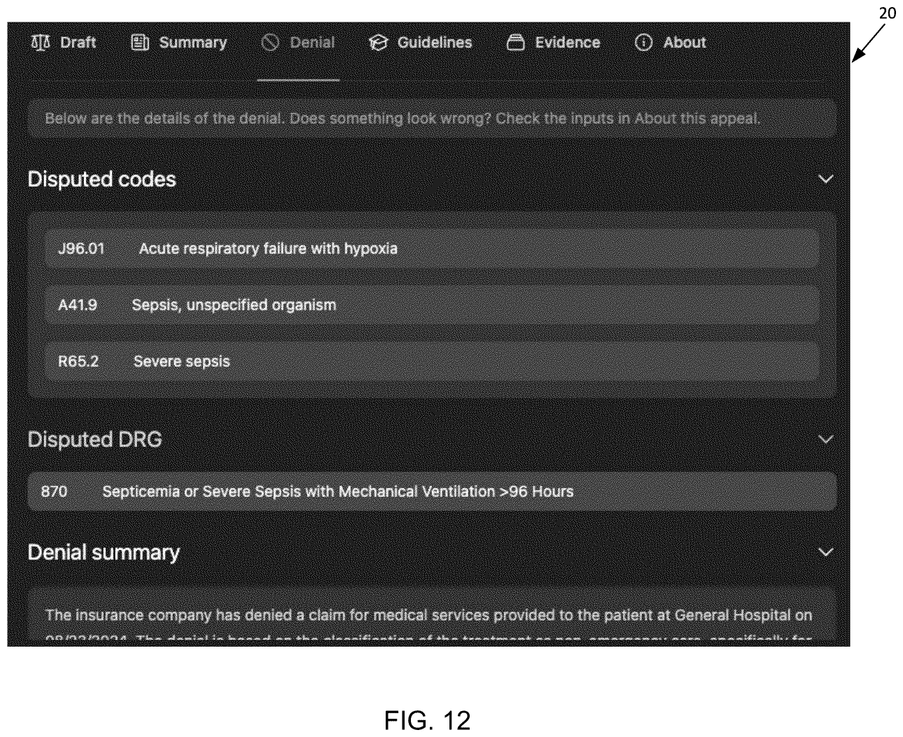

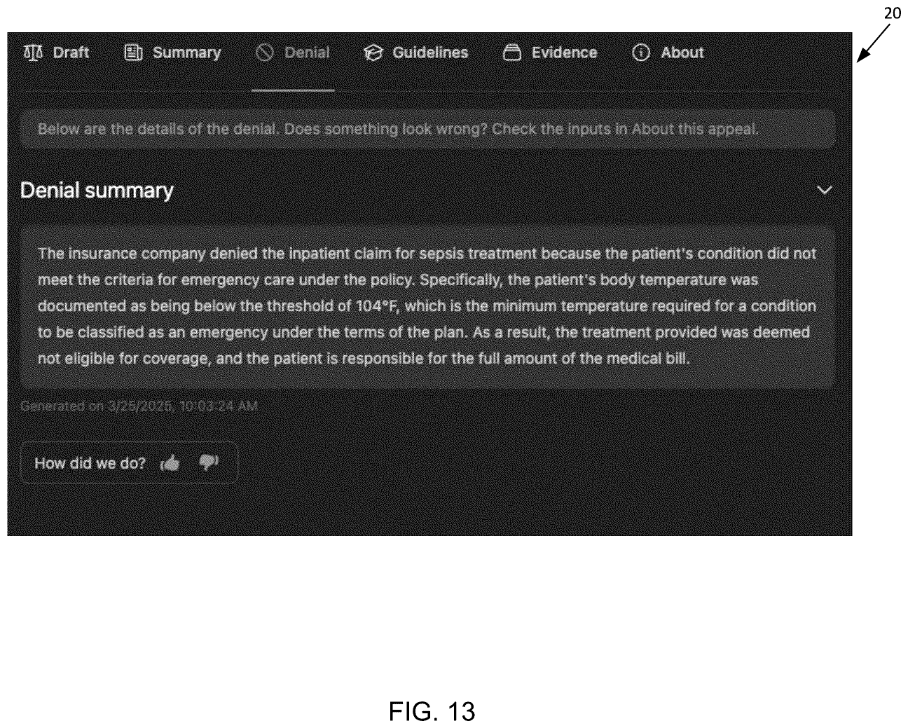

FIGS. 12 and 13 show screenshots of the Denial tab 1006 of the example user interface. Depending on your denial type, the Denial tab 1006 displays a summary of the denial letter provided, the initial summary note, or disputed diagnosis codes and the disputed DRG. In one or more arrangements, diagnostic guidelines DRG Downgrades are directly sourced from ACDIS PRO. These guidelines provide accurate reference data to better assist the user in drafting and/or revising the appeal letter. For each disputed code, the user can see the ACDIS guideline outlining the criteria.

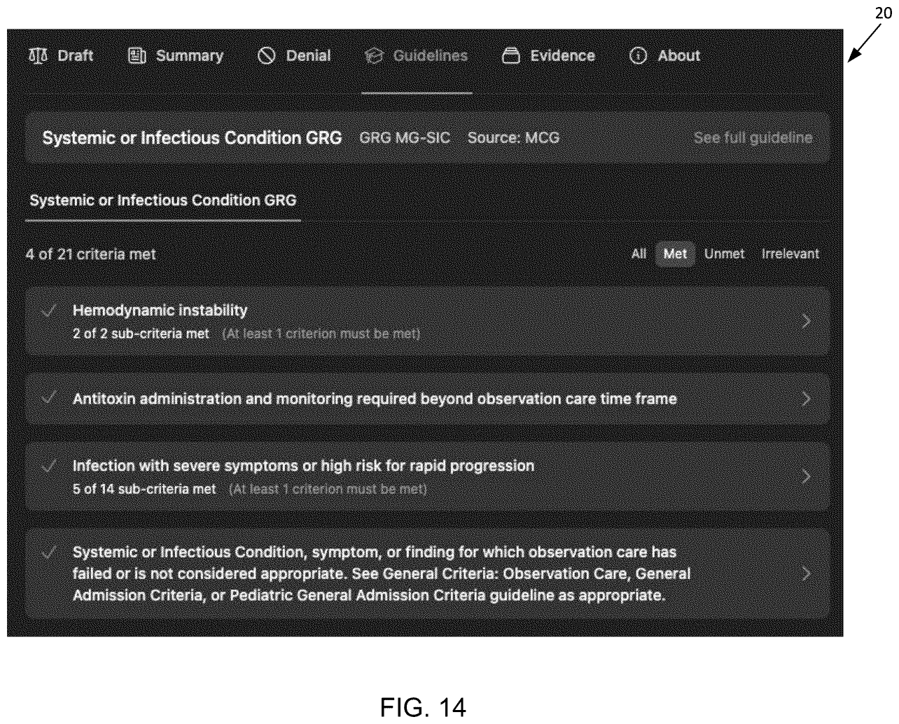

FIG. 14 shows a screenshot of the Guidelines tab 1008 of the example user interface 20.

The Guidelines tab 1008 shows medical necessity and authorization appeal guidelines. In one or more arrangements, the guidelines are sourced directly from MCG, focusing on the guidelines for Inpatient admission. In one or more arrangements, Guidelines tab 1008 displays a hierarchy tree to show if the criteria was met, unmet, or irrelevant. It will also provide citations to show how it was met or reasoning as to why it was not met.

In one or more arrangements, user interface 20 is configured to automatically rearrange the listed guidelines to assist a user in more quickly assessing which guidelines should be used for the appeal. For example, in one or more arrangements, guidelines may be ranked based on satisfied criteria and probability of success for appeal (e.g., according to which guidelines best support the diagnoses and/or procedures related to the reasons for denial). In one or more arrangements, user interface 20 is configured to dynamically reassess and adjust the arrangement of guidelines in response to changes/corrections made by a user in review of records and evidence. The automated rearrangement of guidelines can help avoid the need of a user to scroll through and evaluate the entire list of guidelines to best to use for appeal.

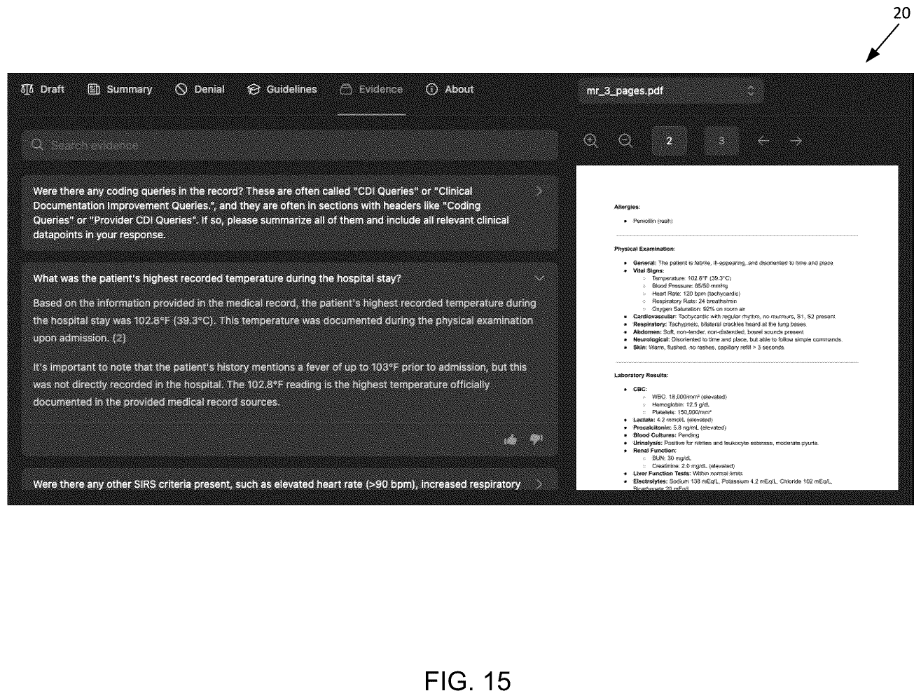

FIG. 15 shows a screenshot of the Evidence tab 1010 of the example user interface 20. The Evidence tab 1010 provides the user a picture into every question or query that was asked to evaluate the denial and the case. In one or more arrangements, Evidence tab 1010 shows the question, the response, and provides citations so the user can check the work performed by system 10. In one or more arrangements, a user can view the question and answer pairs to validate the information presented in the summary. The answers will provide page links to where the system found the answers.

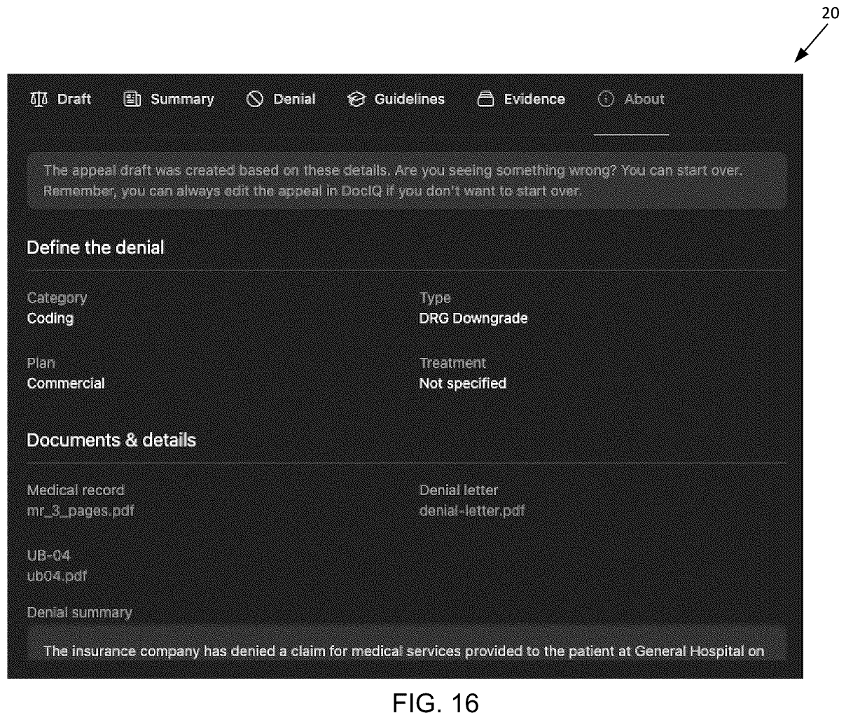

FIG. 16 shows a screenshot of the About tab 1012 of the example user interface 20. The About tab 1012 allows a user to view the information that was input into the system 10 for evaluation of the denied benefit system claim and/or generation of an appeal letter.

From the above discussion, it will be appreciated that system 10 improves upon the state of the art. For example, some various embodiments provide an improved system for processing and/or review of documents for denied benefit system claims and/or generation of appeals letters to petition for reconsideration. It will be appreciated by those skilled in the art that other various modifications could be made to the device without parting from the spirit and scope of this disclosure. All such modifications and changes fall within the scope of the claims and are intended to be covered thereby.

What is claimed:

1. A system, comprising:
a front end system;
a back end system;
the back end system communicatively connected to the front end system;
wherein the back end system is configured to perform one or more processes to automatically review documents for a denied benefit system claim and automatically generate a letter to petition for reconsideration;
wherein the front end system is configured to provide a user interface for a user to submit a denial letter for the denied benefit system claim and prompt back end system to perform the one or more processes to generate the letter to petition for reconsideration.

2. The system of claim 1, wherein the one or more processes performed by the back end system includes:
evaluating a denial of a benefit coverage claim and determining one or more reasons for the denial;
retrieving guidelines and/or policies relating to applicability of the one or more reasons for the denial;
retrieving and processing medical records to gather evidence in support of the petition for reconsideration;
generation of arguments for the letter;
the arguments including arguments for a clinical determination and/or justification for failure to follow procedural requirements; and
generating the letter based on the generated arguments.

3. The system of claim 1, wherein the one or more processes performed by the back end system includes performing a clinical evaluation by:
evaluating a denial letter for the denied benefit system claim to identify a set of diagnoses and/or procedures related to the denial;
determining a set of criteria corresponding to the diagnoses and/or procedures of the set of diagnoses or procedures;
ranking the determined set of criteria;
evaluating the set of criteria in ranked order to identify one of the set of criteria that is satisfied.

4. The system of claim 1, wherein the one or more processes performed by the back end system includes performing a procedural evaluation by:
summarizing procedures that were followed from administration records;
based on the summarized procedures and a denial letter for the denied benefit system claim, determining if the benefit system claim was denied because a procedure was not followed;
in response to identifying the procedure that was not followed, generating a justification for the procedure not being followed.

5. The system of claim 1, wherein the one or more processes performed by the back end system includes:
evaluating the generated letter for a set of quality and compliance measures and determining an evaluation score;
in response to the evaluation score being greater than or equal to a predetermined threshold causing the generated letter to be submitted for appeal of the denied benefit system claim;
in response to the evaluation score being below the predetermined threshold, causing the generated letter to be forwarded for human review vis the user interface.

6. The system of claim 1, wherein the back end system includes a clinical evaluation subsystem, a procedural evaluation subsystem, a content retrieval subsystem, an appeal letter generation subsystem, and a quality and compliance subsystem.

7. A system, comprising:
a back end system;
the back end system communicatively connected to a front end system;
wherein the back end system is configured to perform one or more processes to automatically review documents for a denied benefit system claim and automatically generate a letter to petition for reconsideration.

8. The system of claim 7, further comprising a front end system communicatively connected to the backend system;
wherein the front end system is configured to provide a user interface for a user to submit a denial letter for the denied benefit system claim and prompt back end system to perform the one or more processes to generate the letter to petition for reconsideration.

9. The system of claim 7, further comprising a front end system communicatively connected to the backend system;
wherein the one or more processes performed by the back end system includes:
evaluating the generated letter for a set of quality and compliance measures and determining an evaluation score;
in response to the evaluation score being greater than or equal to a predetermined threshold causing the generated letter to be submitted for appeal of the denied benefit system claim;
in response to the evaluation score being below the predetermined threshold, causing the generated letter to be forwarded for human review vis a user interface provided by the front end system.

10. The system of claim 7, wherein the one or more processes performed by the back end system includes:
evaluating a denial of a benefit coverage claim and determining one or more reasons for the denial;
retrieving guidelines and/or policies relating to applicability of the one or more reasons for the denial;
retrieving and processing medical records to gather evidence in support of the petition for reconsideration;
generation of arguments for the letter;
the arguments including arguments for a clinical determination and/or justification for failure to follow procedural requirements; and
generating the letter based on the generated arguments.

11. The system of claim 7, wherein the one or more processes performed by the back end system includes performing a clinical evaluation by:
evaluating a denial letter for the denied benefit system claim to identify a set of diagnoses and/or procedures related to the denial;
determining a set of criteria corresponding to the diagnoses and/or procedures of the set of diagnoses or procedures;
ranking the determined set of criteria;
evaluating the set of criteria in ranked order to identify one of the set of criteria that is satisfied.

12. The system of claim 7, wherein the one or more processes performed by the back end system includes performing a procedural evaluation by:
summarizing procedures that were followed from administration records;
based on the summarized procedures and a denial letter for the denied benefit system claim, determining if the benefit system claim was denied because a procedure was not followed;
in response to identifying the procedure that was not followed, generating a justification for the procedure not being followed.

13. The system of claim 7, wherein the back end system includes a clinical evaluation subsystem, a procedural evaluation subsystem, a content retrieval subsystem, an appeal letter generation subsystem, and a quality and compliance subsystem.

14. A system, comprising:
a clinical evaluation subsystem;
wherein the clinical evaluation subsystem is configured to:
evaluate a denial letter for a denied benefit system claim to identify a set of diagnoses and/or procedures related to the denial;
determine a set of criteria corresponding to the diagnoses and/or procedures of the set of diagnoses or procedures;
rank the determined set of criteria;
evaluate the set of criteria in ranked order to identify one of the set of criteria that is satisfied;
a procedural evaluation subsystem;
wherein the procedural evaluation subsystem is configured to summarize procedures that were followed from administration records;
based on the summarized procedures and a denial letter for the denied benefit system claim, determine if the benefit system claim was denied because a procedure was not followed; and
in response to identifying the procedure that was not followed, generate a justification for the procedure not being followed;
an appeal letter generation subsystem;
wherein the appeal letter generation subsystem is configured to generate an appeal letter to petition for reconsideration of the denied benefit system claim based on the one of the set of criteria identified by the clinical evaluation subsystem and/or the justification generated by the procedural evaluation subsystem.

15. The system of claim 14, wherein the appeal letter generation subsystem is configured to assess applicability of a set of conditional arguments using a large language model (LLM) to identify a set of applicable arguments;
wherein the appeal letter generation subsystem is configured to generate the appeal letter with the set of applicable arguments using the LLM.

16. The system of claim 14, further comprising a quality and compliance subsystem;
wherein the quality and compliance subsystem is configured to evaluate and score the generated appeal letter.

17. The system of claim 14, further comprising a quality and compliance subsystem;
wherein the quality and compliance subsystem is configured to evaluate the generated appeal letter for a set of quality and compliance measures and determining an evaluation score;
wherein in response to the evaluation score being greater than or equal to a predetermined threshold the quality and compliance subsystem is configured to cause the generated letter to be submitted for appeal of the denied benefit system claim;
in response to the evaluation score being below the predetermined threshold, the quality and compliance subsystem is configured to cause the generated letter to be forwarded for human review vis a user interface.

18. The system of claim 14, further comprising a content retrieval subsystem;
wherein the content retrieval subsystem is configured parse documents related to the denied benefit system claim to populate a searchable database for use by the clinical evaluation subsystem, the procedural evaluation subsystem, and/or the appeal letter generation subsystem.

19. The system of claim 14, further comprising a content retrieval subsystem;
wherein the content retrieval subsystem is configured parse documents related to the denied benefit system claim to populate a searchable database having content of the documents separated into content segments and having metadata describing the content segments.

20. The system of claim 14, further comprising a content retrieval subsystem;
wherein the content retrieval subsystem is configured parse documents related to the denied benefit system claim to populate a searchable database having content of the documents separated into content segments and having metadata describing the content segments;
wherein in response to receiving a query requesting information, the content retrieval subsystem is configured retrieve the information from the database by retrieving a set of segments from the database that have metadata matching the query and ranking the set of segment to determine the most relevant segments in the set of segments.
